(19) United States
(12) Plant Patent Application Publication
(10) Pub. No.: US 2019/0357398 P1
(43) Pub. Date: Nov. 21, 2019

(54) Euonymus plant named 'HEESGO1801'

(50) ;

(71) Applicant: Van Heesbeen Cultures B.V., Rijsbergen (NL)

(72) Inventor: Ad van Heesbeen, Rijsbergen (NL)

(21) Appl. No.: 16/501,669

(22) Filed: May 20, 2019

(30) Foreign Application Priority Data
May 18, 2018 (QZ) 2018/1337

(51) Int. Cl. A01H 6/00

(52) U.S. Cl. PLT/246

(57) ABSTRACT

A new and distinct Euonymus plant named ‘HEESGO1801’ which is characterized by the combination of a large plant size, globular habit, yellow to green-yellow foliage, and the stability of these characteristics from generation to generation.

### CROSS REFERENCE TO RELATED APPLICATIONS

This application claims priority to the Community Plant Variety Rights application number 2018/1337, filed May 18, 2018, which is herein incorporated by reference.

Latin name of the genus and species: The Latin name of the genus and species of the novel variety disclosed herein is Euonymus japonicus.

Variety denomination: The inventive cultivar of Euonymus disclosed herein has been given the variety denomination ‘HEESGO1801’.

### BACKGROUND OF THE INVENTION

Parentage: ‘HEESGO1801’ originated as a naturally-occurring, whole plant mutation of an unnamed Euonymus japonicus plant (not patented). In June of 2016 the inventor discovered the mutation at a commercial greenhouse in Rijsbergen, the Netherlands, growing amongst a cultivated population of unnamed Euonymus japonicus plants. The mutation was noted for its unique foliage characteristics and was subsequently isolated for further evaluation in order to confirm the distinctness and stability of the characteristics first observed. Upon confirmation of distinctness and stability, the new Euonymus plant was selected for commercialization and given the name ‘HEESGO1801’.

Asexual Reproduction: Asexual reproduction of ‘HEESGO1801’, by way of softwood stem cuttings, was first performed in June of 2016 in Rijsbergen, the Netherlands. Through five subsequent generations, the unique features of this cultivar have proven to be stable and true to type.

### SUMMARY OF THE INVENTION

The cultivar ‘HEESGO1801’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘HEESGO1801’. These characteristics in combination distinguish ‘HEESGO1801’ as a new and distinct Euonymus japonicus cultivar:
1. Euonymus ‘HEESGO1801’ exhibits a relatively large plant size; and
2. Euonymus ‘HEESGO1801’ exhibits a globular plant shape; and
3. Euonymus ‘HEESGO1801’ exhibits light green-yellow stems; and
4. Euonymus ‘HEESGO1801’ exhibits foliage ranging in color from yellow to green-yellow.

### BRIEF DESCRIPTION OF THE FIGURES

FIG. 1 illustrates, as nearly true as it is reasonably possible to make the same in color photographs of this type, an exemplary mature plant of ‘HEESGO1801’ grown outdoors in Rijsbergen, the Netherlands.

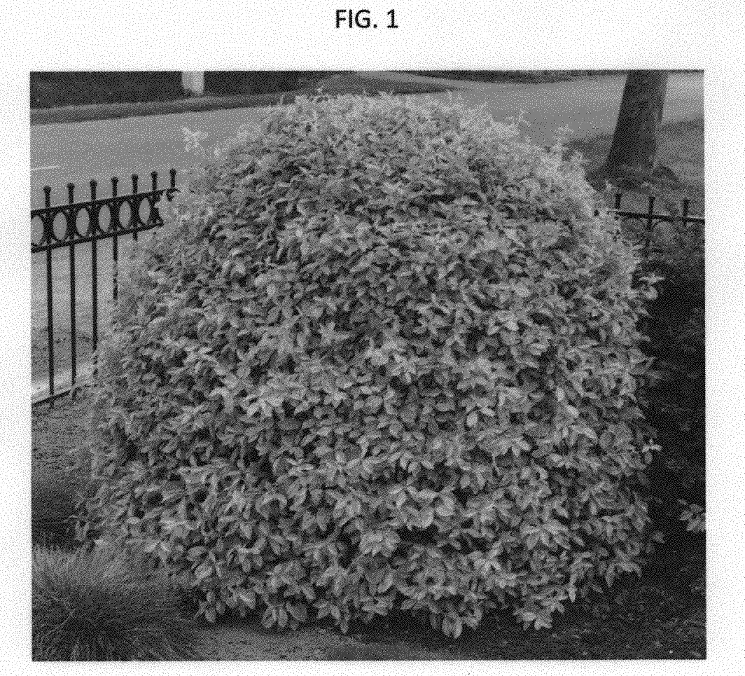

FIG. 2 illustrates, as nearly true as it is reasonably possible to make the same in color photographs of this type, the typical foliage of ‘HEESGO1801’.

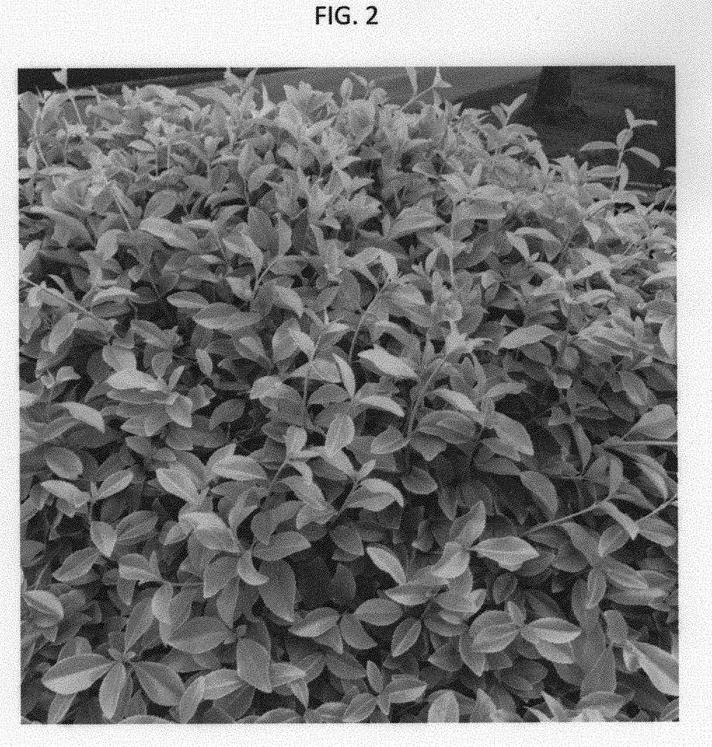

### BOTANICAL DESCRIPTION OF THE PLANT

A detailed botanical description of a new and distinct variety of Euonymus plant known as ‘HEESGO1801’ is not yet available. A comparison with the parents and most similar variety of common knowledge are provided below.
General plant description: Botanical description is not yet available.
Root system: Botanical description is not yet available.
Stem: Botanical description is not yet available.
Foliage: Botanical description is not yet available.
Inflorescence: Botanical description is not yet available.
Flower buds: Botanical description is not yet available.
Flower: Botanical description is not yet available.
Reproductive organs: Botanical description is not yet available.
Seed and Fruit: Botanical description is not yet available.

### COMPARISONS WITH THE PARENTS

Plants of the new cultivar ‘HEESGO1801’ differ from its parent, Euonymus japonicus ‘Heespierrolino’ (U.S. Plant Pat. No. 13,852), by the characteristics described in Chart 1.

CHART 1

| Characteristic | ‘HEESGO1801’ | ‘Heespierrolino’ |
|---|---|---|
| Foliage size. | Smaller than | Larger than ‘HEESGO1801’. |
| | ‘Heespierrolino’. | |
| Foliage margin. | Coarsely denticulate. | Finely denticulate. |
| General coloration of | Medium green. | A combination of yellow and |
| mature foliage. | | green-yellow. |

### COMPARISONS WITH THE MOST SIMILAR VARIETY OF COMMON KNOWLEDGE

Plants of the new cultivar ‘HEESGO1801’ may be distinguished from the commercial variety Euonymus japonicus ‘BR2012’ (Community Plant Variety Rights application number 2016/0820) by the combination of characteristics described in Chart 2.

CHART 2

| Characteristic | ‘HEESGO1801’ | ‘BR2012’ |
|---|---|---|
| Growth habit. | Larger; less compact. | Smaller; more compact. |
| Mature foliage; | Entire plant. | Towards the apex. |
| distribution of yellow- | | |
| colored foliage. | | |

That which is claimed is:

1. A new and distinct cultivar of Euonymus plant named ‘HEESGO1801’, substantially as described and illustrated herein.
